(19) United States
(12) Patent Application Publication
(10) Pub. No.: US 2021/0355097 A1
(43) Pub. Date: Nov. 18, 2021

(54) CATALYTIC CONVERSATION OF CANNABIDIOL AND METHODS THEREOF

(71) Applicant: FinChemica, Inc., Superior, CO (US)

(72) Inventor: Christopher C.F. Cooper, Superior, CO (US)

(21) Appl. No.: 17/315,500

(22) Filed: May 10, 2021

Related U.S. Application Data
(60) Provisional application No. 63/023,647, filed on May 12, 2020.

(51) Int. Cl. C07D 311/78; B01J 27/128; B01J 31/02; B01J 31/30

(52) U.S. Cl. CPC C07D 311/78; B01J 31/30; B01J 31/0232; B01J 27/128

(57) ABSTRACT

A method of converting cannabidiol (CBD) into Δ9-Tetrahydrocannabinol (Δ9-THC) and Δ8-Tetrahydrocannabinol (Δ8-THC). The method provides a polar aprotic solvent such as Tert-Butyl Methyl Ether, Tetrahydrofuran, dicloromethane, or chloroform. Cannabidiol starting material mixes into the polar aprotic solvent in a chemical reactor to make a cannabinoid solution. Adding a metallic catalyst capable of performing intramolecular hydroalkoxylation to the cannabinoid solution and mixing it converts the cannabidiol starting material into Δ9-Tetrahydrocannabinol (Δ9-THC) and Δ8-Tetrahydrocannabinol (Δ8-THC) in a ratio of at least 6:1. The catalyst is a metal such as a transition metal or is selected from the group consisting of ruthenium, aluminum, iron, gold, silver, copper, platinum, and combinations thereof. In one embodiment a co-catalyst is used such as a triflate salt. Regulating the temperature of the reaction to less than 20° C. yields a predominance of Δ9-THC, i.e. Δ9-THC is more than 75% of the cannabinoid mix.

### FIELD OF THE INVENTION

This invention relates to methods of converting cannabidiol into Δ9-tetrahydrocannabinol.

### BACKGROUND OF THE INVENTION

Cannabis Sativa L. is the binomial name for a plant species that includes both hemp and marijuana in common language usage. Marijuana is typically a craft crop grown indoors, or in greenhouses. Occasionally it is grown outdoors in small plots. Marijuana plant biomass has a greater tetrahydrocannabinol (THC) content than does Hemp plant biomass.

Hemp is typically an industrially farmed crop and is defined by law to have a maximum of 0.3% THC in the U.S., and a maximum of 1% THC in some countries outside of the U.S.

Hemp varieties include some that produce over 20% cannabidiol (CBD) content in the flowering biomass but has very little THC. Due to regulatory restrictions on growing marijuana, and the industrial manner under which Hemp may be farmed, the production costs of CBD are generally much less than that of equal amounts of TIC.

What is desired is a way of producing THC that is less expensive than traditional agricultural methods. What is also desired is a way of utilizing an agricultural commodity such as CBD to product THC in a safe, clean, and cost effective way.

WO2009099868A1 to Trawick et al. discloses the chemical synthesis of Δ9-tetrahydrocannabinol (Δ9-THC) and related compounds. In particular, the process comprises a one-pot condensation and sulfonylatton reaction sequence that produces crude Δ9-=THC aryl sulfonate or related compounds. Sulfonylation of Δ9-THC or related compounds immediately upon their formation imparts stability to the cannabinoids, and prevents formation of the corresponding Δ8 isomer. Δ9THC aryl sulfonates may be readily separated from Δ6THC aryl sulfonates using reverse phase chromatography. Hydrolysis of the Δ9-THC aryl sulfonates or related compounds produces Δ9-THC or related compounds containing relatively low amounts of the corresponding Δ8 isomer.

U.S. Pat. No. 8,106,244B2 to Burdick et al. discloses a process for preparation of a delta-9-tetrahydrocannabinol compound or derivative thereof involving treating a first intermediate compound with an organoaluminum-based Lewis acid catalyst, under conditions effective to produce the delta-9-tetrahydrocannabinol compound or derivative thereof.

The structure of CBD consists of a terminal olefin that is in very close proximity to a phenol. While there are a myriad of ways to do this transformation, most of them suffer from secondary reactions taking place in other areas of the CBD molecule. Previous attempts in the literature to perform this transformation typically use acid catalysis (strong acids —either Bronstead or Lewis), which makes the formation of the product in reasonable selectivity challenging. Prior attempts convert CBD via strong acids such as sulfuric acid into a product having predominately Δ8THC, which is not a commercially desirable product.

Prior attempts of conversion yielded a ratio of Δ9THC:Δ8THC in the ballpark of 2:1 under optimized conditions. This is a useful advancement in the art from a scientific standpoint but is certainly not nearly efficient or optimal for industrial implementation of such processes.

These and other early methods of CBD to THC conversion lack the ability to control the production desired isomers of the THC molecule with optimal yields and efficiency demanded by commercial and industrial processes.

### SUMMARY OF THE INVENTION

The present invention uses transition metal catalysis in order to selectively convert cannabidiol (CBD) into a desired isoforms of Tetrahydrocannabinol (THC). Preferably, the CBD is derived from hemp and is used to produce, in a desired quantity, or ratio, Δ8 or Δ9-tetrahydrocannabinol (Δ8 or Δ9 THC), or both.

While the ability to convert CBD to these products has been known, the challenge has been in developing techniques that will selectively convert to a large majority of Δ8 or Δ9. The present invention does both.

Use of at least one transition metal catalyst capable of promoting hydroalkoxylation on an olefin is preferred in one embodiment of the present invention.

The structure of CBD consists of a terminal olefin that is in very close proximity to a phenol. While there are a myriad of ways to do this transformation, most of them suffer from secondary reactions taking place in other areas of the CBD molecule. Previous attempts in the literature to perform this transformation typically use acid catalysis (strong acids —either Bronstead or Lewis), which makes the formation of the product in reasonable selectivity challenging. Early attempts of conversion yielded a ratio of Δ9-THC:Δ8-THC of 2:1 under optimized conditions.

By using transition metal catalysis at a relatively low temperature i.e. below 20° C., the ratio of Δ9-THC:Δ8-THC can be pushed in excess of 6:1, with final Δ9-THC percentages being at least 75% and typically greater than 80%, and the total content of the product (total cannabinoid percentage) being greater than 95%.

Critical process parameters: The catalyst being used can be ruthenium, aluminum, iron, gold, silver, copper, or platinum based—or any other metal based catalyst capable of performing intramolecular hydroalkoxylation. Thus, the intramolecular hydroalkoxylation catalyst is selected from the group consisting of rutherium, aluminum, iron, gold, silver, copper, platinum and combinations thereof. In the case of combinations of such catalysts, sequential processes using single catalysts are possible to maximize yield, also a combination of catalysts can be used simultaneously. Analogues, salts, isoforms, ions, and variations of the catalysts listed are contemplated for use with the present invention.

In some instances, a co-catalyst triflate salt is added in order to increase reactivity. From a practical standpoint, iron based catalysts are the preferred embodiment due to their ease of availability, ease of handling, low toxicity, and low cost. The amount of catalyst used can be anywhere between 1 and 99% on a mol basis relative to the starting material. In practice, about a 15 mol % loading of catalyst is sufficient for proper conversion.

The solvent used for the process can be any solvent that CBD is soluble in. Preferred solvents are aprotic solvents with a low polarity (for example, TBME, THF, DCM, chloroform). However, highly polar solvents can also be used (nitromethane, acetonitrile) While it will work in a wide array of solvents, the preferred solvent is TBME (tertiary-butyl methyl ether).

The amount of solvent can be anywhere from 1 volume relative to the weight of the starting material to 100 volumes. The preferred amount is about 5 volumes.

The temperature at which the reaction runs determines the ratio of Δ9-THC:Δ8-TIC observed in the final material. Lower temperatures favor the formation of Δ9-THC, whereas higher temperatures favor the formation of Δ8-THC. At temperatures below 20° C., the final concentration of Δ9-THC formed is greater than 75% in the isolated products.

As this is producing THC distillate, it can be used as an immediate replacement to any formulation that uses traditional THC distillate in it. This eliminates the need for indoor grow facilities, and allows one to source their starting material easily.

### BRIEF DESCRIPTION OF THE DRAWINGS

FIG. 1 is a chemical reaction converting CBD to Tetrahydrocannabinol (THC) with the FeCl, catalyst.

FIG. 2 is a flow chart of a method in accordance with the present invention.

### DETAILED DESCRIPTION

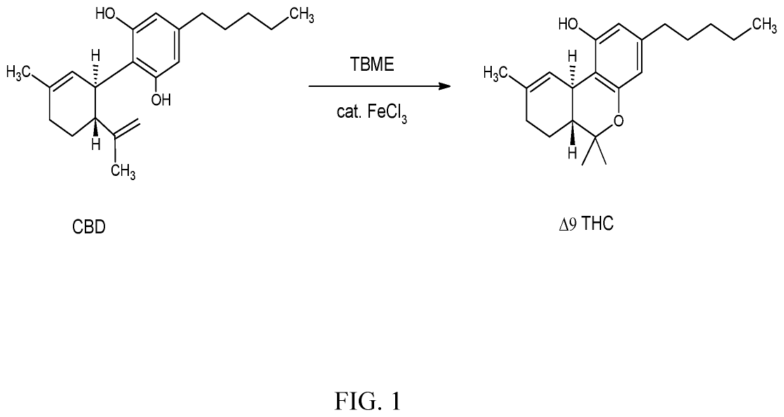

FIG. 1 shows a chemical reaction converting CBD to Δ9 Tetrahydrocannabinol (THC) with the FeCl₃ catalyst. Preferably, the reaction converts cannabidiol (CBD) into Δ9-Tetrahydrocannabinol (Δ9-THC) and detectable amounts of Δ8-Tetrahydrocannabinol (Δ8-THC).

A method of the invention provides a polar aprotic solvent such as Tert-Butyl Methyl Ether, Tetrahydrofuran, dicloromethane, or chloroform. Cannabidiol starting material mixes into the polar aprotic solvent in a chemical reactor to make a cannabinoid solution. Adding a metallic catalyst capable of performing intramolecular hydroalkoxylation to the cannabinoid solution and mixing it converts the cannabidiol into Δ9-Tetrahydrocannabinol (Δ9-THC) and Δ8-Tetrahydrocannabinol (Δ8-THC) in a ratio of at least 6:1. The catalyst is a metal such as a transition metal or is selected from the group consisting of ruthenium, aluminum, iron, gold, silver, copper, platinum, and combinations thereof: In one embodiment a co-catalyst is used such as a triflate salt. Regulating the temperature of the reaction to less than 20° C. yields a predominance of Δ9-THC, i.e. Δ9-THC is more than 75% of the cannabinoid mix.

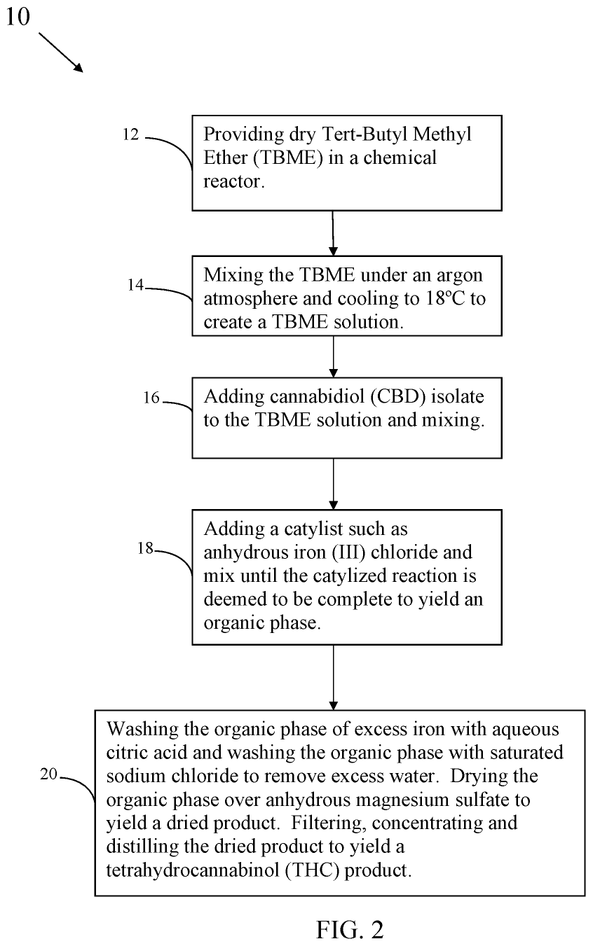

FIG. 2 shows a method of the present invention generally designated with the reference numeral 10. The method 10 converts CBD isolate to Δ9 THC using a FeCl₃ catalyst and a saturated hydrocarbon solvent. Preferably this process yields at least 80% Δ9 THC in the cannabinoid mix, and a product having a greater than 95% cannabinoid content. The ratio of Δ9 THC:Δ8 THC is greater than 6:1.

The term THC as used herein includes the combination of acid form and non-acid forms of Tetrahydrocannabinol, as well as isoforms thereof unless the isoforms are particularly specified such as Δ9-Tetrahydrocannabinol (Δ9-THC) or Δ8-Tetrahydrocannabinol (Δ8-THC).

The term “cannabinoid mix” is the mixture of cannabinoids in a sample of biomass, distillate, isolate, formulation, or other cannabinoid rich product. The term “cannabinoid” encompasses hundreds of bioactive compounds and molecules commonly found in Cannabis saliva L that are proven to influence or impact the CB1, CB2, 5-HT1A, TRPV1, GPR55, PPARs or other receptors in the human endocannabinoid system.

Influence can be up regulation, down regulation or modulation of the particular cannabinoid receptor, including allosteric modulation. Cannabinoids can act as a receptor antagonist, agonist or combination thereof depending on many factors including the presence of other cannabinoids.

The method 10 includes the step 12 of providing dry Tert-Butyl Methyl Ether (TBME) in a chemical reactor. The step 12 can utilize any solvent that can be used with cannabidiol (CBD). Alternatively, various aprotic solvents with a low polarity can be used. For example Tetrahydrofuran (THF), DCM, chloroform, and analogs thereto can be used in accordance with the present invention. Step 12 simply sets forth a preferred solvent.

In an alternate embodiment of step 12, a polar solvent can be used. For example, polar solvents such as nitromethane and acetonitrile may be used.

It can be appreciated that various hydrocarbon solvents including Pentane and Hexane Hydrocarbon analogues of TBME can be used in accord with the present invention. For example, an alternative to TBME can be the Hexane Tert-Butyl Ethyl Ether C₆H₁₄O under varied circumstances.

The amount of solvent used can vary from 1 to 100 molecular volumes per weight of the starting material. Preferably, the amount of solvent used it is about 5 volumes. Preferably, the starting material is cannabidiol (CBD) isolate having a purity of at least 95% in a dry powdered form.

The step 14 mixes the TBME Mixing the TBME under an argon atmosphere and cooling to 18° C. to create a TBME solution, the step 16 of adding cannabidiol (CBD) isolate to the TBME solution and mixing, the step 18 of adding a catalyst such as anhydrous iron (III) chloride and mix until the catalyzed reaction is deemed to be complete to yield an organic phase, the method 10 includes the step 20 of washing the organic phase of excess iron with aqueous citric acid and washing the organic phase with saturated sodium chloride to remove excess water. The step 20 further includes drying the organic phase over anhydrous magnesium sulfate to yield a dried product. Filtering, concentrating and distilling the dried product to yield a tetrahydrocannabinol (THC) product.

The step 12 includes providing a clean 20 L chemical reactor. The step 14 adds 10 L of a dry saturated hydrocarbon such as a pentanol hydrocarbon.

Preferably the saturated hydrocarbon is Tert-Butyl Methyl Ether (TBME) having the chemical formula C₅H₁₂O and a molecular weight (MW) of 88.15 g/mol. The step 14 mixes the TBME under a non-reactive atmosphere, such as an inert gas atmosphere. Preferably the atmosphere is an argon atmosphere.

One advantage of using TBME is the boiling point of approximately 131.4° F. (55° C.), which makes utilization in liquid phase viable across a broad working temperature range.

The step 14 cools the TBME in the argon atmosphere to 18° C., which is below its' boiling point.

The step 16 adds 2.0 kg (6.36 mol, 1.0 Eq) of cannabidiol isolate to the cooled TBME. Mix until dissolution is complete. Next, in step 18, the method adds a catalyst.

In step 18, preferably the catalyst is 30 g (0.18 mol, 0.0283 equivalent weight (Eq)) of anhydrous iron (III) chloride having the chemical formula FeCl₃ to the reactor and stirs the catalyst and the cannabinoid solution for between 20 minutes to 40 minutes, preferably for 30 minutes. FeCl₃ has a molecular weight of 162.204 g/mol. In an alternate embodiment the catalyst range of 0.1 mol to 3 mol is utilized.

During the step 18, progress of the reaction is observed via utilizing high pressure liquid chromatography (HPLC) (Restek Raptor ARC-18 HPLC column). The step 18 adds additional 30 g (0.184952282311164 mol) charges of the iron catalyst in 30 minute increments until virtually all of the cannabidiol starting material has been converted i.e. until the reaction is deemed to be complete by the disappearance of starting material. In practice, this typically takes about 120 g (0.739809129244656 mol) of catalyst (0.12 Eq total equivalents) and at least two hours of time. Eq=MW/n wherein “Eq” is the equivalent weight, which is the molecular weight (MW) divided by the number of equivalents (n).

Once the reaction is deemed to be complete, excess iron is removed via washing the organic phase with aqueous citric acid. The organic phase is also washed with saturated sodium chloride to remove excess water. The organic phase is dried over anhydrous magnesium sulfate, is filtered, and is concentrated under reduced pressure to yield the product in the form of a brown oil. This oil is then further purified via distillation, to yield 1.8 kg in the form of a pale yellow oil with a purity of at least 95% delta 9 THC.

Benefits of the FeCl₃ catalyst is that the cost is low compared to other catalysts available. The reaction is predictably efficient, consistent even with temperature variations and low toxicity in any final product.

1. A method for converting cannabidiol (CBD) into Δ9-Tetrahydrocannabinol (Δ9-THC) and Δ8-Tetrahydrocannabinol (Δ8-THC), the method comprising:
providing a solvent;
mixing cannabidiol starting material and the solvent in a chemical reactor to make a cannabinoid solution;
adding a catalyst to the cannabinoid solution and mixing to convert the cannabidiol starting material into Δ9-Tetrahydrocannabinol (Δ9-THC) and Δ8-Tetrahydrocannabinol (Δ8-THC), wherein the temperature at which the catalyst is mixed with the cannabinoid solution determines the ratio of Δ9-Tetrahydrocannabinol (Δ9-THC) conversion and Δ8-Tetrahydrocannabinol (Δ8-THC) conversion.

2. The method as set forth in claim 1, wherein the cannabidiol starting material is cannabidiol isolate with at least a 95% purity.

3. The method as set forth in claim 2, wherein the cannabidiol starting material includes cannabidiol and at least one other cannabinoid and having less than 95% purity.

4. The method as set forth in claim 2, wherein the catalyst is anhydrous iron (III) chloride.

5. The method as set forth in claim 2, wherein preferably the catalyst is anhydrous iron (III) chloride and the method stirs the catalyst and the cannabinoid solution for between 20-40 minutes, preferably 30 minutes.

6. The method as set forth in claim 5, wherein the step of adding a catalyst repeats in preferably 30 minutes increments until virtually all of the cannabidiol starting material has been converted.

7. The method as set forth in claim 6, wherein progress of the conversion reaction is observed via utilizing high pressure liquid chromatography.

8. The method as set forth in claim 6, wherein the amount of catalyst used is between 1% and 99% on a molecular percentage basis.

9. The method as set forth in claim 6, wherein the amount of catalyst used is 15% on a molecular percentage basis.

10. The method as set forth in claim 5, wherein the process yields a product having at least 95% cannabinoid content.

11. The method as set forth in claim 5, wherein the process yields a product having at least 80% Δ9-Tetrahydrocannabinol (Δ9-THC).

12. The method as set forth in claim 5, wherein the catalyst converts a portion of the cannabidiol starting material into tetrahydrocannabinol to yield a product having at least 95% cannabinoid content including detectable amounts of tetrahydrocannabinol.

13. The method as set forth in claim 1, wherein the temperature at which the catalyst is mixed with the cannabinoid solution remains above 20° C. to favor conversion of the cannabidiol starting material into Δ8-Tetrahydrocannabinol (Δ8-THC) conversion compared to conversion of the cannabidiol starting material into Δ9-Tetrahydrocannabinol (Δ9-THC).

14. The method as set forth in claim 1, wherein the temperature at which the catalyst is mixed with the cannabinoid solution remains below 20° C. to favor conversion of the cannabidiol starting material into Δ9-Tetrahydrocannabinol (Δ9-THC) conversion compared to conversion of the cannabidiol starting material into Δ8-Tetrahydrocannabinol (Δ8-THC).

15. The method as set forth in claim 1, wherein the temperature at which the catalyst is mixed with the cannabinoid solution remains below 20° C. to favor conversion of cannabidiol starting material into Δ9-Tetrahydrocannabinol (Δ9-THC) and the method yields a product having a cannabinoid mix with at least 75%, typically greater than 80%, Δ9-Tetrahydrocannabinol (Δ9-THC).

16. The method as set forth in claim 1, wherein the temperature at which the catalyst is mixed with the cannabinoid solution remains below 20° C. to favor conversion of cannabidiol starting material into Δ9-Tetrahydrocannabinol (Δ9-THC) and the method yields a product having a cannabinoid mix with at least 80% Δ9-Tetrahydrocannabinol (Δ9-THC).

17. (canceled)

18. A method of converting cannabidiol (CBD) into Δ9-Tetrahydrocannabinol (Δ9-THC) and Δ8-Tetrahydrocannabinol (Δ8-THC), the method comprising:
providing a polar aprotic solvent selected from the group consisting essentially of Tert-Butyl Methyl Ether, Tetrahydrofuran, dicloromethane, chloroform, and combinations thereof;
mixing cannabidiol starting material and the polar aprotic solvent in a chemical reactor to make a cannabinoid solution;
adding a metallic catalyst capable of performing intramolecular hydroalkoxylation to the cannabinoid solution and mixing to convert the cannabidiolstarting material into Δ9-Tetrahydrocannabinol (Δ9-THC) and Δ8-Tetrahydrocannabinol (Δ8-THC) in a Δ9-THC:Δ8-THC ratio of at least 6:1.

19. The method as set forth in claim 18, wherein the catalyst is selected from the group consisting essentially of ruthenium, aluminum, iron, gold, silver, copper, or platinum.

20. The method as set forth in claim 18, wherein the catalyst is a transition metal catalyst.

21. The method as set forth in claim 18, wherein the catalyst further comprises a triflate salt co-catalyst.
